(19) United States
(12) Patent Application Publication
(10) Pub. No.: US 2008/0046007 A1
(43) Pub. Date: Feb. 21, 2008

(54) Surgical fastener

(75) Inventors: Richard F. Schwemberger, Cincinnati, OH (US); John P. Measamer, Cincinnati, OH (US); Michael S. Cropper, Edgewood, KY (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Ethicon Endo-Surgery, Inc.

(21) Appl. No.: 11/507,117

(22) Filed: Aug. 21, 2006

(51) Int. Cl. A61B 17/08

(52) U.S. Cl. 606/220

(57) ABSTRACT

A surgical fastener for fastening tissue. The fastener includes a first piece having a curved needle, a second piece having an opening sized for receiving the needle, and a lock for holding the needle of the first piece in the opening of the second piece.

### BACKGROUND OF THE INVENTION

The present invention relates generally to a surgical fastener, and more particularly to a surgical fastener having curved piercing elements.

Gastroesophageal reflux disease or persistent heartburn is caused by an improper relaxation of the lower esophageal sphincter, allowing acidic stomach contents to travel into the esophagus. If left untreated, chronic reflux may cause esophageal stricture, bleeding ulcers, perforation, and scarring. Continued reflux may lead to Barrett's esophagus, involving changes in the esophageal cells and possibly leading to cancer. Antacids and proton pump inhibitors are initially used to treat this condition. If these treatments are unsuccessful, surgical intervention is often recommended.

One interventional surgical method is known as Nissen fundoplication. This procedure involves wrapping a fundus of the stomach around the lower end of the esophagus and fastening it in place to make the lower esophageal sphincter less compliant. Traditionally, this procedure was accomplished by open surgery using sutures to secure the plicated fundus of the stomach around the esophagus without penetrating the stomach. More recently, laparoscopic Nissen procedures have been used. In some laparoscopic procedures, surgical fasteners are used with an endoscopic applicator. Several different fastener designs have been developed. Some of these designs include a two piece fastener. A first of these pieces includes a base having two straight elongate needles extending perpendicularly outward from the base generally parallel to each other. A second piece includes a receiver element having openings positioned for receiving the needles of the first piece and a lock for holding the needles in place once received in the openings. In use, tissue is gathered, the needles of the first piece are pushed through the gathered tissue and the openings of the second piece to hold the tissue and fastener in place. U.S. Pat. No. 7,033,378 discloses one such fastener and an applicator used with this fastener.

The applicator disclosed in U.S. Pat. No. 7,033,378 includes an elongate shaft having two jaws pivotally attached to its end. The jaws include receptacles for holding the first and second pieces of the fastener. The jaws push the needles of the first piece through the gathered tissue and the openings of the second piece to hold the tissue and fastener in place. As will be appreciated by those skilled in the art, because the jaws are pivotally attached to the shaft, each of the needles sweeps through an arc as the jaws pivot to pierce the tissue and push the needles into the corresponding openings. As a result of the needles sweeping through arcs, the needles do not enter and pass through the tissue at a single point. Rather, the needles trace a line, increasing the force required to push the needles through the tissue and potentially causing the tissue to tear.

Moreover, on occasion fasteners will become unfastened during post-operative recovery so the fasteners are free to move about the patient's body. In the case of fasteners used in gastroesophageal applications, the unfastened fasteners can move through the patient's esophagus, stomach and/or bowel. As the fasteners move, the piercing elements of can pierce portions of the patient's body with which they come in contact unless provisions are made to protect the body from the elements.

### SUMMARY OF THE INVENTION

Briefly, the present invention includes a surgical fastener for fastening tissue. The fastener comprises a first piece having a curved needle, a second piece having an opening sized for receiving the needle therein, and a lock for holding the needle of the first piece in the opening of the second piece.

In another aspect, the invention includes a surgical fastener for fastening tissue. The fastener comprises a first piece having a curved needle, and a second piece having an opening sized for receiving the needle and adapted for holding the needle of the first piece in the opening of the second piece.

In yet another aspect, the invention includes a surgical fastener for fastening tissue. The fastener comprises a first piece having a base and a plurality of curved needles attached thereto. Each of the needles is adapted for movement between a collapsed position in which the needle extends generally parallel to the base and a deployed position in which the needle extends outward from the base. The fastener also comprises a second piece having a plurality of openings therein. Each of the openings is sized and positioned for receiving one of the needles therein when the needles are in the deployed position. Further, the fastener comprises a lock for holding each of the needles of the first piece in the respective opening of the second piece.

In another aspect, the invention includes a surgical fastener for use with an applicator to fasten tissue. The fastener comprises a first piece including a needle having a shape defined be a path through which the applicator moves the needle when fastening tissue. In addition, the fastener comprises a second piece having an opening sized for receiving the needle therein, and a lock for holding the needle of the first piece in the opening of the second piece.

Other features of the present invention will be in part apparent and in part pointed out hereinafter.

### BRIEF DESCRIPTION OF THE DRAWINGS

FIG. 1 is a perspective of a surgical fastener of the present invention;

FIG. 2 is a perspective of a male piece of the fastener of FIG. 1 shown with needles in a deployed position;

FIG. 3 is a perspective of the male piece shown with the needles in a collapsed position;

FIG. 4 is an alternate perspective of the surgical fastener;

FIG. 5 is a perspective similar to FIG. 1 with a latch body cover and base removed to facilitate viewing interior structure;

FIG. 6 is a perspective of a surgical fastener of the present invention shown with a portion of an applicator used to install the fastener in a patient;

FIG. 7 is a perspective similar to FIG. 1 with a slide assembly in a locked position; and

FIG. 8 is a perspective similar to FIG. 5 with the slide assembly in the locked position.

Corresponding reference characters indicate corresponding parts throughout the several views of the drawings.

### DETAILED DESCRIPTION OF THE PREFERRED EMBODIMENT

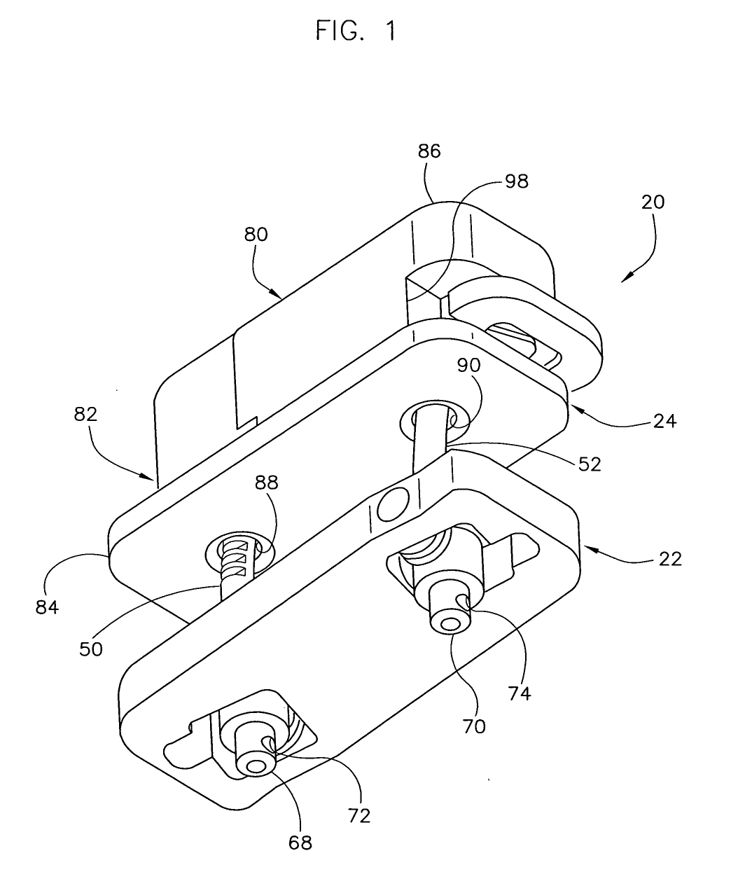

Referring now to the drawings and in particular to FIG. 1, a two-piece fastener of the present invention is designated in its entirety by the reference numeral 20. The fastener 20 includes a male piece, generally designated 22, and a female piece, generally designated 24.

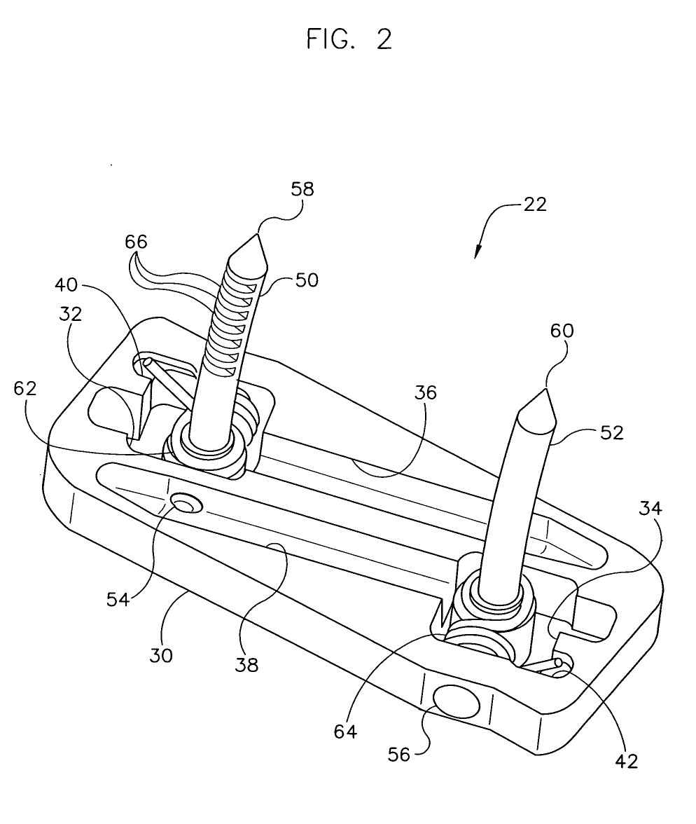

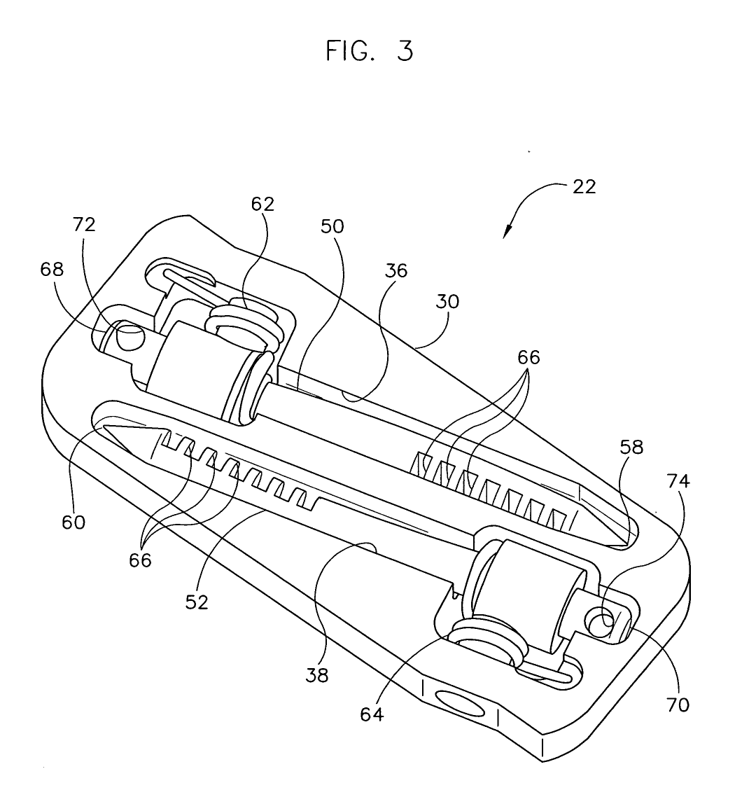

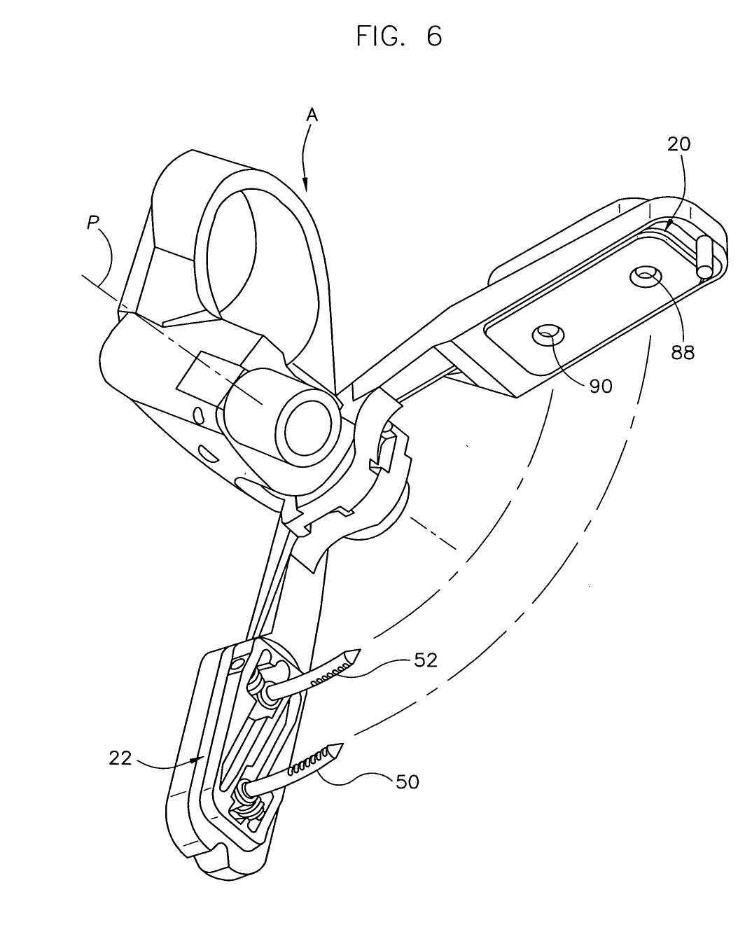

As shown in FIG. 2, the male piece 22 includes a base 30 having two openings 32, 34. An elongate channel 36, 38 extends from each opening 32, 34. A recess 40, 42 is positioned adjacent each opening 32, 34, generally opposite one of the channels 36, 38. Two elongate curved needles or posts 50, 52 are rotatably coupled to the base 30. Each of the needles 50, 52 is held in position in its corresponding opening 32, 34 by an axle pin 54, 56 so the needle is aligned with its corresponding channel 36, 38. Although the needles 50, 52 are adapted to pierce tissue, in one embodiment they have slightly rounded tips 58, 60 so they displace tissue rather than cut tissue. Torsion springs 62, 64 are fitted over each axle pin 54, 56 so one end is coupled to the corresponding needle 50, 52 and another end is biased against the base 30 in the corresponding recess 40, 42. The torsion springs 62, 64 operate to bias the corresponding needle 50, 52 toward a collapsed position in which the needles lie in the corresponding channel 36, 38 as shown in FIG. 3. Thus, when the male piece 22 and the female piece 24 are unfastened, the needles 50, 52 collapse so the tips 58, 60 are covered and cannot pierce tissue within the patient as the fastener 20 moves through the body. When the needles 50, 52 extend generally perpendicular to the base as shown in FIG. 2, the needles are in a deployed position. Although the needles 50, 52 may have other lengths departing from the scope of the present invention, in one embodiment each needle has a length measured from a center of the axle pin to its respective tip of between about ten millimeters and about twenty millimeters. In one particular embodiment, each needle 50, 52 has a length of at least about twenty millimeters so it can penetrate two full thicknesses of stomach tissue (each full thickness including a mucosal layer, a muscularus layer and a serosal layer). Although the needles 50, 52 may have other shapes and dimensions without departing from the scope of the present invention, in one embodiment each needle has a circular cross section and a diameter of between about one millimeter and about two millimeters. In one particular embodiment, each needle 50, 52 tapers from a diameter of less than about one millimeter at the tip to less than 1.5 millimeters at the base so the needles penetrate tissue easily, so the needles have sufficient strength to maintain alignment during tissue penetration and so the holes made by the needles in the tissue are not prone to leakage. Further, although the needles 50, 52 may have other shapes without departing from the scope of the present invention in one embodiment the needles are curved. In one particular embodiment, the needles 50, 52 are arcuate and have a radius of curvature of between about twelve millimeters and about thirty millimeters. In one embodiment, the radius of curvature of each needle 50, 52 is equal for manufacturing convenience. In another embodiment shown in FIG. 6, the needles 50, 52 have a common center of curvature C and radii of curvature corresponding to a distance between the center of curvature and the particular needle. As will be appreciated by those skilled in the art, the common center of curvature in this embodiment is positioned at the pivot axis P of jaws of an applicator A which are used to install the fastener in the patient. One such applicator A is described in U.S. Pat. No. 7,033,378 and will not be described in further detail. In one embodiment, the needles 50, 52 lie in a common plane.

As further shown in FIG. 3, the channels 36, 38 may be oriented at an angle within the base 30 to accommodate longer needles 50, 52 without increasing a length of the base. Each needle 50, 52 includes a series of notches or grooves 66 near its tip 58. 60. A lower portion 68, 70 of each needle 50, 52 opposite the tip 58, 60 extends beyond the axle pin 54, 56 (FIG. 2) so that the lower portion extends beyond the base 30 when the needles are in the deployed position as shown in FIG. 2. The lower portions 68, 70 may be held by an applicator (not shown) to maintain the needles 50, 52 in the deployed position against the forces applied by the torsion springs 62, 64. The lower portions 68, 70 may also include openings 72, 74 for receiving portions of the applicator. Features of an exemplary applicator are described in detail in U.S. Pat. No. 7,033,378.

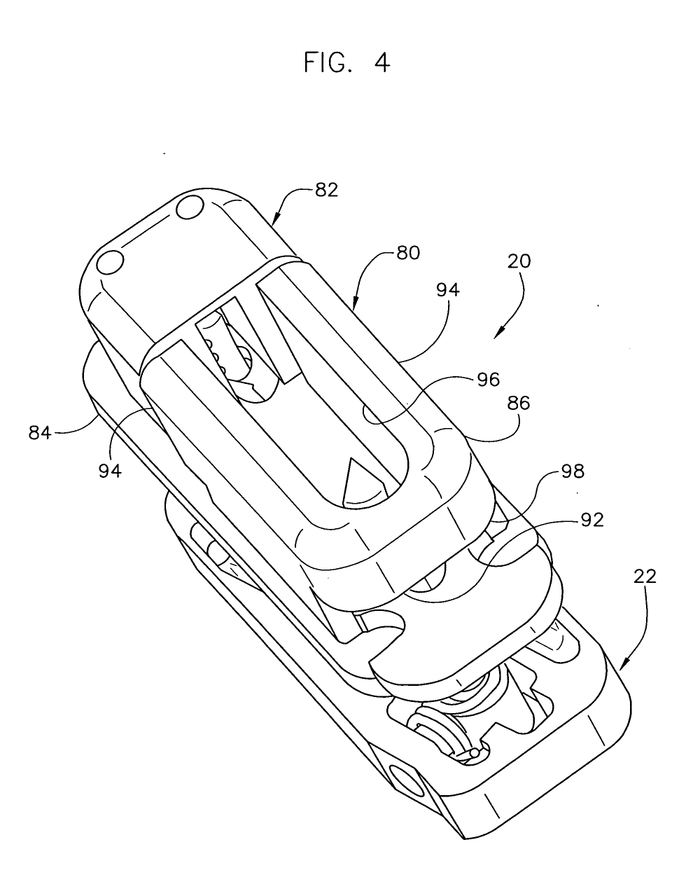

As shown in FIG. 1, the female piece 24 includes a latch body, generally designated by 80, and a slide assembly, generally designated by 82, slidably connected to the latch body. The latch body 80 includes a base 84 and a cover 86. The base 84 includes two openings or holes 88, 90. These holes 88, 90 are sized and positioned to receive the needles 50, 52 of the male piece 22. Preferably, the holes 88, 90 are chamfered to guide the needles 50, 52 into them to permit modest misalignment of the needles and holes. As illustrated in FIG. 4, in one embodiment the cover 86 of the latch body is U-shaped, having an end portion 92 and two side portions 94 extending from the end portion. Thus, the end portion 92 and side portions 94 define a central opening 98 in the cover 86. The end portion 92 has an opening 96 adjacent the base 84 of the latch body 80 for accommodating a portion of the slide assembly 82 as will be explained in greater detail below.

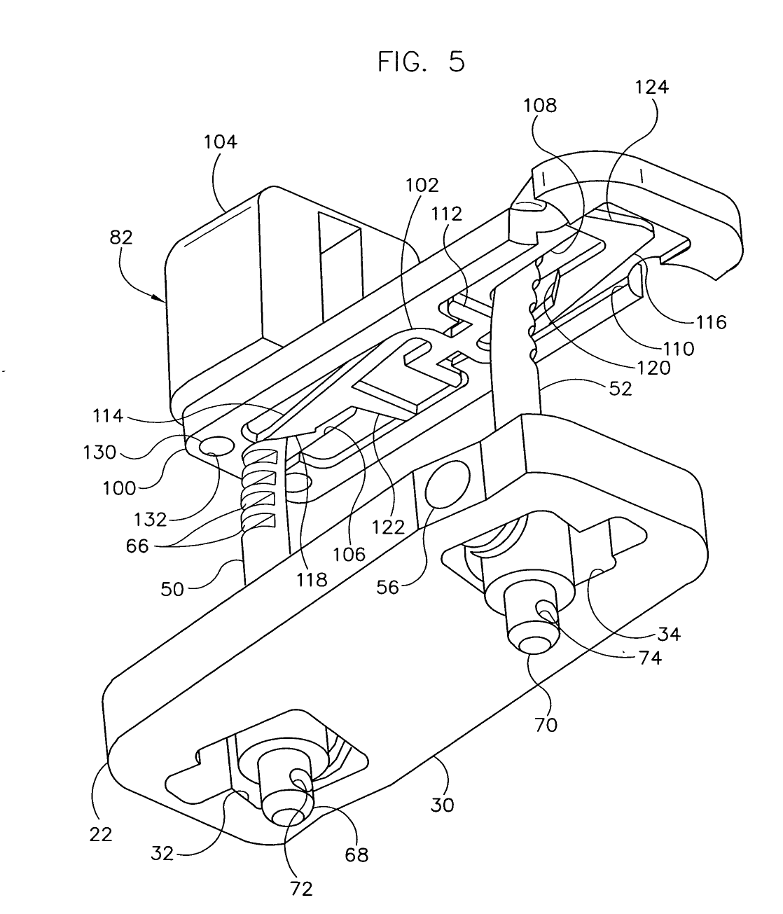

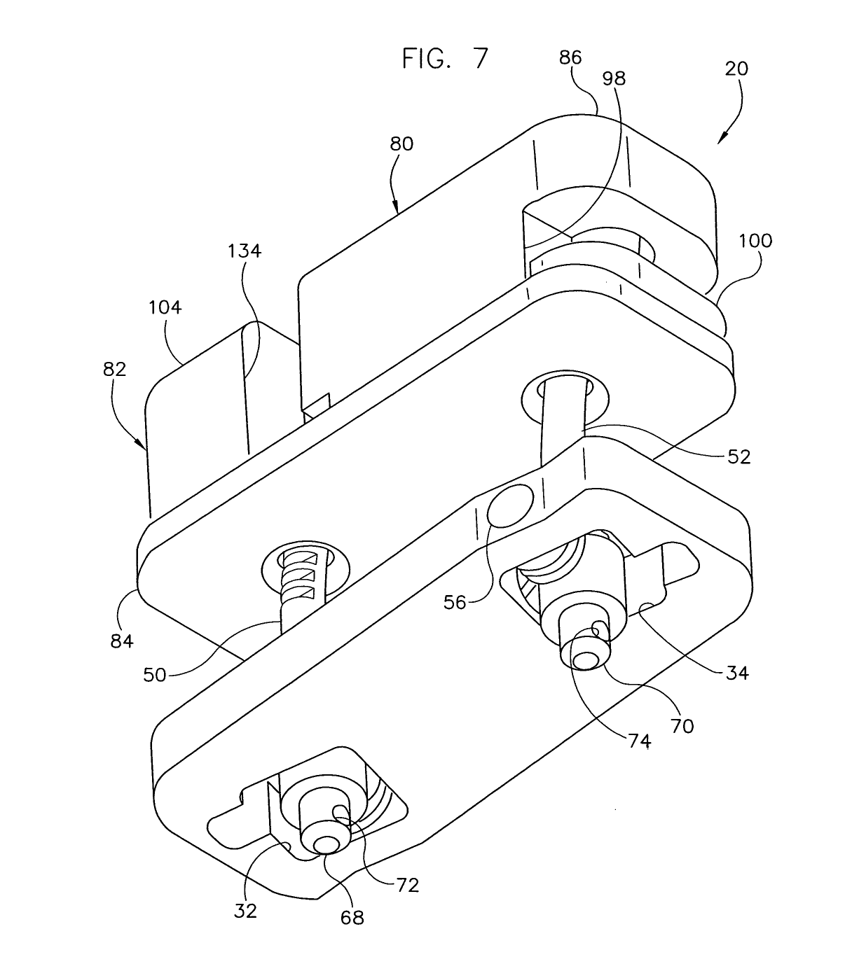

As illustrated in FIG. 5, the slide assembly 82 includes a body 100, a lock 102, and a cover 104. The body 100 has two elongate slots 106, 108 sized and positioned for receiving the needles 50, 52 when extending through the holes 88, 90 in the base 84 of the latch body 80. As will be appreciated by those skilled in the art, the slots 106, 108 permit the slide assembly 82 to move relative to the latch body 80 between an unlocked position shown in FIG. 1 and a locked position shown in FIG. 7. As further shown in FIG. 5, the body 100 also has a recess 110 for receiving the lock 102. The lock 102 includes a central portion 112 sized and shaped for engaging the recess of the body 100 to hold the lock in position relative to the body. As will be appreciated by those skilled in the art, the lock 102 is captured between the slide assembly body 100 and the base 84 of the latch body 80 so the lock is retained in the recess 110. Two arms or catches 114, 116 extend outward in opposite directions from the central portion 112 of the lock 102. The arms 114, 116 are flexible but biased toward a position in which they overlap the slots 106, 108 in the body 100. As will be described below, each arm 114, 116 selectively engages corresponding slots 66 in the needles 50, 52. Each of the arms 114, 116 includes an angled portion 118, 120 for guiding the respective arm into the corresponding slot. The arms 114, 116 also include stops 122, 124 for preventing the arms from blocking the slots 106, 108 when the needles 50, 52 are not present. During manufacture, the slide assembly cover 104 is attached to the latch slide 100 with pins 130 that are press fit into coupling holes 132, and the lock 102 is inserted into the recess 110. The slide assembly 82 is then captured between the latch body 80 and base 84.

Although the fastener 20 may have other dimensions without departing from the scope of the present invention, in one embodiment the fastener sized for passing through a patient's esophagus. For example, in one embodiment the male piece 22 has a length of about 15 mm, a width of about 6.25 mm, and a height of about 2 mm when the needles are in their collapsed positions. In this embodiment, the female piece 24 has a length of about 15 mm, a width of about 6.25 mm, and a height of about 4 mm. The coupled fastener 20 of this embodiment has a length of about 15 mm, a width of about 6.25 mm, and a height of about 6 mm plus the thickness of the tissue captured between the male and female pieces.

Although the pieces 22, 24 may be made of other materials without departing from the scope of the present invention, in one embodiment the pieces are made of titanium or titanium alloy, and anodized. In one particular embodiment, the pieces 22, 24 are anodized to have a color that is distinct from natural colors of tissue of the stomach cavity so that the pieces may be easily seen during visual inspection during surgery. Preferred colors include purple, blue and black.

A conventional instrument (not shown) such as described in U.S. Pat. No. 7,033,378 may be used to install the fastener 20 of the present invention. The male and female pieces 22, 24 of the fastener 20 are positioned on opposite sides of tissue to be fastened. The instrument holds the needles 50, 52 of the male piece 22 in the deployed position as shown in FIG. 2 so the needles can pierce the tissue and extend into the holes 88, 90 of the base 84 of the female piece 24 (FIG. 1). The chamfered openings of the holes 58, 60 guide the needles 50, 52 into the holes if the pieces 22, 24 are slightly misaligned. As will be appreciated by those skilled in the art, the needles 50, 52 travel along arcs as the applicator pushes the needles through the tissue and into the holes 88, 90 in the base 84 of the female piece 24. Because the arcs through which the needles 50, 52 travel correspond to their overall shape, the needles follow their respective points 58, 50 and do not stretch or tear the tissue as they travel along their arced path. When the needles 50, 52 are inserted in the holes 88, 90 in the base, the slide cover 104 and latch cover 86 cover the sharp points 58, 60 of needles so they will not penetrate or injure adjacent tissue in the patient.

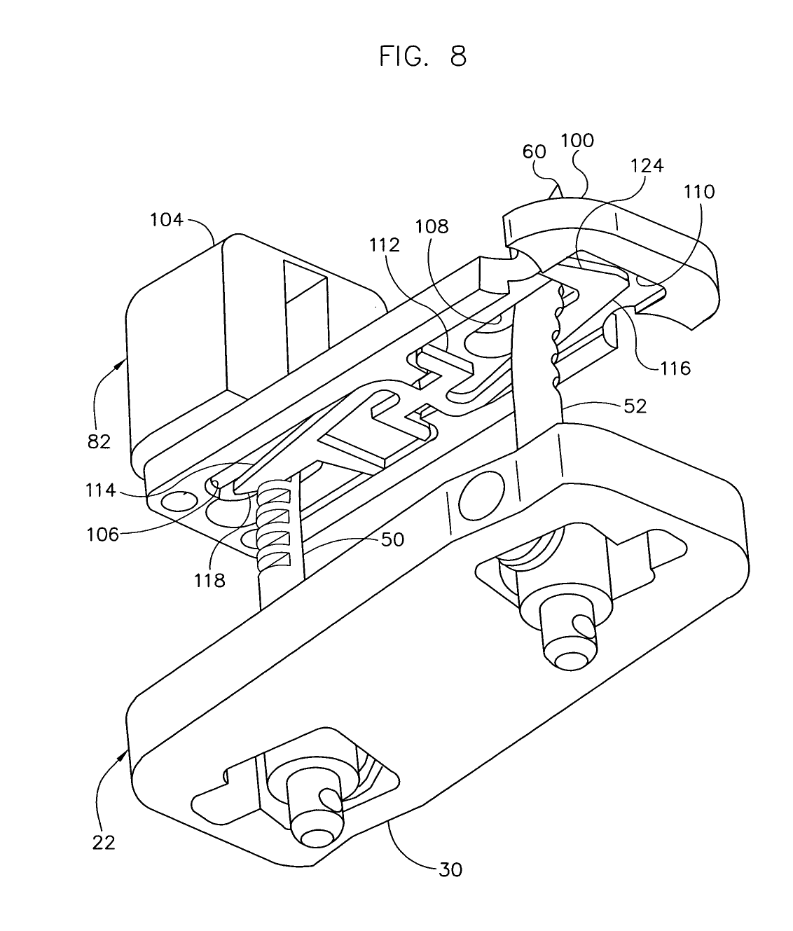

Referring now to FIGS. 7 and 8, once the fastener 20 is positioned around the tissue with a desired clamping force (or desired pressure), the slide assembly 82 is moved longitudinally relative to the latch body 80 until the body 100 abuts the cover 86 of the latch body 80. As the slide assembly 82 moves relative to the body 80, the arms 114, 116 engage the respective slots 66 of the needles 50, 52, thereby locking the male and female pieces 22, 24 together and clamping the captured tissue between them. The plurality of slots 66 permit the base 30 of the male piece 22 and the base 84 of the latch body 80 to be spaced at several different distances relative to each other when the fastener 20 is fastened to tissue. Moreover, the bases 30, 84 may be angled relative to each other so that one needle is inserted farther into the latch body than the other needle. This permits the fastener 20 to accommodate various configurations of tissue. As a result of this adjustability, a desired force can be applied to tissue between the pieces 22, 24 whether or not the tissue has a uniform thickness. Accordingly, force can be limited to prevent tissue necrosis. When the slide assembly 82 is moved relative to the latch body 80, the arms 114, 116 will automatically find an appropriate slot 66. Further, the flexibility of the arms 114, 116 compensates for small amounts of misalignment between the arms and slots 66. If an arm 114, 116 of the lock 102 initially contacts a needle 50, 52 at a non-slotted location, the flexibility of the arm will permit it to move into an adjacent slot 66. When locked, a gap 134 is formed between the latch body 80 and the cover 86 of the slide assembly 82 as shown in FIG. 7.

After the male and female pieces 22, 24 have been locked together, they may be unlocked and separated from each other. To unlock the fastener 20, the slide assembly 82 is moved relative to the latch body 80 so the gap 134 closes. As the gap 134 closes, the arms 114, 116 disengage the slots 66 so the pieces 22, 24 can be pulled apart. It is envisioned that the fastener 20 can be unlocked, separated and retrieved from the patient using a standard endoscopic snare device (not shown). The snare device is looped around the slide body 104 and cover 86. As the snare loop is pulled, the snared parts are pulled toward each other by decreasing the size of the snare loop. As discussed above, the needles 50, 52 are spring-biased to collapse into the channels 36, 38 of the base 30 of the male piece 22 when not retained against the bias of the torsion springs 62, 64. This operates to prevent injury to the patient when the pieces 22, 24 are unlocked and separated, or if the male piece 22 inadvertently becomes separated from the applicator or from the female piece 24. Given the size of the pieces 22, 24 and the protection of sharps from exposure to the patient, the pieces may be safely passed through the gastrointestinal system.

It is recognized that various other configurations can be used for locking the female piece 24 relative to the needles 50, 52 of the male piece 22. Examples of alternative configurations are included in U.S. Pat. No. 7,033,378.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A surgical fastener for fastening tissue, said fastener comprising:
a first piece having a curved needle;
a second piece having an opening sized for receiving said needle therein; and
a lock for holding the needle of said first piece in the opening of said second piece.

2. A surgical fastener as set forth in claim 1 wherein said first piece includes a base and said needle extends from said base.

3. A surgical fastener as set forth in claim 2 wherein said needle is pivotally attached to said base for movement between a collapsed position in which the needle extends generally parallel to the base and a deployed position in which the needle extends outward from the base.

4. A surgical fastener as set forth in claim 3 wherein said needle is biased toward the collapsed position.

5. A surgical fastener as set forth in claim 4 further comprising a spring operatively connected between the needle and the base for biasing the needle toward the collapsed position.

6. A surgical fastener as set forth in claim 4 wherein the base includes a channel for receiving the needle when in the collapsed position.

7. A surgical fastener as set forth in claim 1 wherein said needle includes a groove and said lock engages the groove to hold the needle of said first piece in the opening of said second piece.

8. A surgical fastener as set forth in claim 7 wherein said lock includes an arm that engages the groove in the needle to hold the needle of said first piece in the opening of said second piece.

9. A surgical fastener as set forth in claim 8 wherein said arm is biased toward the needle when the needle of said first piece is inserted in the opening of said second piece.

10. A surgical fastener as set forth in claim 1 wherein:
the needle of the first piece moves through an arc as it is received in the opening of the second piece; and
the needle includes an arcuate portion having a radius of curvature corresponding to the arc traced by the needle.

11. A surgical fastener for fastening tissue, said fastener comprising:
a first piece having a curved needle; and
a second piece having an opening sized for receiving said needle and adapted for holding the needle of said first piece in the opening of said second piece.

12. A surgical fastener as set forth in claim 11 wherein:
the needle of the first piece move through an arc as it is received in the opening of the second piece; and
the needle includes an arcuate portion having a radius of curvature corresponding to the arc traced by the needle.

13. A surgical fastener for fastening tissue, said fastener comprising:
a first piece having a base and a plurality of curved needles attached thereto, each of said plurality of needles being adapted for movement between a collapsed position in which the needle extends generally parallel to the base and a deployed position in which the needle extends outward from the base;
a second piece having a plurality of openings therein, each of said plurality of openings being sized and positioned for receiving one of said plurality of needles therein when said needles are in the deployed position; and
a lock for holding each of said plurality of needles of said first piece in the respective openings of said second piece.

14. A surgical fastener as set forth in claim 13 wherein each of said plurality of needles is pivotally attached to said base for movement between the collapsed position and the deployed position.

15. A surgical fastener as set forth in claim 14 wherein each of said plurality of needles is biased toward the collapsed position.

16. A surgical fastener as set forth in claim 15 further comprising a plurality of springs, each of said springs being operatively connected between one of said plurality of needles and the base for biasing the respective needle toward its collapsed position.

17. A surgical fastener as set forth in claim 15 wherein the base includes a plurality of channels for receiving each of said plurality of needles when in its collapsed position.

18. A surgical fastener as set forth in claim 13 wherein at least one of said plurality of needles includes a groove and said lock engages the groove to hold the grooved needle in the opening of said second piece.

19. A surgical fastener as set forth in claim 18 wherein each of said needles includes a groove and said lock engages the groove to hold the grooved needle in the opening of said second piece.

20. A surgical fastener as set forth in claim 19 wherein each of said needles includes a plurality of grooves including said aforesaid groove and said lock engages at least one of said plurality of grooves to hold the needle in the opening of said second piece.

21. A surgical fastener as set forth in claim 19 wherein said lock includes an arm that engages the groove in the grooved needle to hold the grooved needle of said first piece in the opening of said second piece.

22. A surgical fastener as set forth in claim 21 wherein said arm is biased toward the grooved needle when the grooved needle of said first piece is inserted in the opening of said second piece.

23. A surgical fastener as set forth in claim 13 wherein:
each of said plurality of needles of the first piece moves through an arc as it is received in the respective opening of the second piece; and
each needle includes an arcuate portion having a radius of curvature corresponding to the arc traced by the respective needle.

24. A surgical fastener for use with an applicator to fasten tissue, said fastener comprising:
a first piece including a needle having a shape defined by a path through which the applicator moves the needle when fastening tissue;
a second piece having an opening sized for receiving said needle therein; and
a lock for holding the needle of said first piece in the opening of said second piece.

25. A surgical fastener as set forth in claim 24 wherein said first piece includes a base and said needle extends from said base.

26. A surgical fastener as set forth in claim 25 wherein said needle is pivotally attached to said base for movement between a collapsed position in which the needle extends generally parallel to the base and a deployed position in which the needle extends outward from the base.

27. A surgical fastener as set forth in claim 26 wherein said needle is biased toward the collapsed position.

28. A surgical fastener as set forth in claim 27 further comprising a spring operatively connected between the needle and the base for biasing the needle toward the collapsed position.

29. A surgical fastener as set forth in claim 27 wherein the base includes a channel for receiving the needle when in the collapsed position.

30. A surgical fastener as set forth in claim 24 wherein said needle includes a groove and said lock engages the groove to hold the needle of said first piece in the opening of said second piece.

31. A surgical fastener as set forth in claim 30 wherein said lock includes an arm that engages the groove in the needle to hold the needle of said first piece in the opening of said second piece.

32. A surgical fastener as set forth in claim 31 wherein said arm is biased toward the needle when the needle of said first piece is inserted in the opening of said second piece.

33. A surgical fastener as set forth in claim 24 wherein the needle includes at least a portion having a curved shape.
